(19) United States
(12) Patent Application Publication
(10) Pub. No.: US 2026/0157797 A1
(43) Pub. Date: Jun. 11, 2026

(54) Optical Plasma Drill

(71) Applicant: InnoVoyce LLC, Boston, MA (US)

(72) Inventors: Richard Shaun Welches, Townsend, MA (US); Stephen Neal Mais, Boston, MA (US)

(21) Appl. No.: 19/413,996

(22) Filed: Dec. 9, 2025

Related U.S. Application Data
(60) Provisional application No. 63/730,381, filed on Dec. 10, 2024.

(51) Int. Cl. A61B 18/22; A61B 18/00

(52) U.S. Cl. CPC A61B 18/22; A61B 2018/00642; A61B 2018/00678; A61B 2018/00708; A61B 2018/00886; A61B 2018/2205

(57) ABSTRACT

The present disclosure regards a laser device that includes a laser source, an optical waveguide, and control circuitry. The laser source is configured to emit a laser beam. The optical waveguide includes proximal and distal ends, and it is positioned to receive the laser beam from the laser source at the proximal end and emit the laser beam at the distal end. The control circuitry is configured to cause the laser source to emit the laser beam. The control circuitry is also configured to detect a plasma ball at the distal end of the optical waveguide. Additionally, the control circuitry is configured to, responsive to detecting the plasma ball, determine an amount of time for stopping the laser beam, and cause the laser source to stop emitting the laser beam for the amount of time and emit the laser beam thereafter.

### TECHNICAL FIELD

The present disclosure relates, generally, to laser technologies and, more specifically, to fiber lasers for use in surgery, biomedicine, and other pertinent fields.

### BACKGROUND

In recent years, fiber optic lasers have revolutionized surgical procedures, offering unprecedented levels of precision and flexibility. Whereas traditional surgical techniques often required large incisions, fiber lasers allow surgeons to minimize tissue trauma during surgery. Additionally, the flexibility and small size of fiber lasers allow surgeons to access to hard-to-reach areas within the body, expanding the scope of potential surgical interventions.

Notwithstanding many advancements in the field, there remain opportunities for further improvements to fiber laser technologies. State-of-the-art surgical lasers still struggle, for instance, with overheating and device failure due to contamination accrued during surgery. Additional development to fiber laser technologies promises to further aid surgeons in conducting surgeries therewith, as well as all others that employ fibers lasers in their respective fields.

### SUMMARY

The present disclosure offers various improvements to current fiber laser technologies, particularly with respect to the use of a laser device as an optical plasma drill. This involves the repeated ignition of contaminants (e.g., tissue, fluids) at the distal end of the optical waveguide of a laser device, which ignition can cause the formation of high-temperature plasma balls. This ignition (esp. plasma balls) is capable of penetrating tissue and most other materials-hence, “optical plasma drill.”

If allowed to burn continuously, the ignited contaminants and/or plasma balls (hereinafter referred to collectively as plasma balls) can damage the laser device or surrounding tissue. Accordingly, the laser device is configured to temporarily terminate laser output after detecting the formation of a plasma ball. This lull in irradiation allows the ball to extinguish and helps to prevent unintended harm. The drilling process continues with the subsequent reactivation of the laser, the formation of another plasma ball, the deactivation of the laser, and so on.

Example embodiments include the following:

A Laser Device. The laser device includes a laser source, an optical waveguide, and control circuitry. The laser source is configured to emit a laser beam. The optical waveguide includes proximal and distal ends, and it is positioned to receive the laser beam from the laser source at the proximal end and emit the laser beam at the distal end. The control circuitry is configured to cause the laser source to emit the laser beam. The control circuitry is also configured to detect a plasma ball at the distal end of the optical waveguide. Additionally, the control circuitry is configured to, responsive to detecting the plasma ball, determine an amount of time for stopping the laser beam, and cause the laser source to stop emitting the laser beam for the amount of time. Further, the control circuitry is configured to cause the laser source to emit the laser beam after the amount of time.

A Non-Transitory, Computer-Readable Medium. The computer-readable medium stores instructions that, when executed by a processor of an electronic device, cause the electronic device to perform operations. The operations include causing a laser source to emit a laser beam. The operations also include detecting a plasma ball at a distal end of an optical waveguide. The optical waveguide includes a proximal end and the distal end, and it is positioned to receive the laser beam from the laser source at the proximal end and emit the laser beam at the distal end. Additionally, the operations include, responsive to detecting the plasma ball, determining an amount of time for stopping the laser beam and causing the laser source to stop emitting the laser beam for the amount of time. Further, the operations include causing the laser source to emit the laser beam after the amount of time.

A Computer-Implemented Method. The method is for operating a laser device as an optical plasma drill, and it includes causing a laser source to emit a laser beam. The method also includes detecting a plasma ball at a distal end of an optical waveguide. The optical waveguide includes a proximal end and the distal end, and it is positioned to receive the laser beam from the laser source at the proximal end and emit the laser beam at the distal end. Additionally, the method includes, responsive to detecting the plasma ball, determining an amount of time for stopping the laser beam and causing the laser source to stop emitting the laser beam for the amount of time. Further, the method includes causing the laser source to emit the laser beam after the amount of time.

A Method of Manufacture. The method is for manufacturing a laser device, and it includes providing a laser source configured to emit a laser beam. The method also includes providing an optical waveguide including proximal and distal ends. Additionally, the method includes positioning the optical waveguide to receive the laser beam from the laser source at the proximal end and emit the laser beam at the distal end. Further, the method includes providing control circuitry configured to cause the laser source to emit the laser beam. The control circuitry is also configured to detect a plasma ball at the distal end of the optical waveguide. Additionally, the control circuitry is configured to, responsive to detecting the plasma ball, determine an amount of time for stopping the laser beam and cause the laser source to stop emitting the laser beam for the amount of time. Further, the control circuitry is configured to cause the laser source to emit the laser beam after the amount of time. Further, the method includes connecting the control circuitry to the laser source such that the control circuitry can cause the laser source to emit the laser beam.

Other configurations of the subject technology will be apparent to those skilled in the art from the detailed description below, which describes various configurations of the subject technology and illustrations thereof. The subject technology is capable of other and different configurations, and its several details are capable of modification in various other respects, all without departing from the scope of the subject technology. Thus, the Drawings and Detailed Description are presented as illustrative in nature and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIGS. 1A and 1B illustrate an example optical waveguide of a laser device configured to operate as an optical plasma drill, according to various aspects of the subject technology.

FIG. 2 illustrates example components of a laser device configured to operate as an optical plasma drill, according to various aspects of the subject technology.

FIGS. 3A-3D illustrate example activity of a laser device operating as an optical plasma drill, according to various aspects of the subject technology.

FIG. 4 illustrates an example process for operating a laser device as an optical plasma drill, according to various aspects of the subject technology.

### DETAILED DESCRIPTION

Introduction. An object of the subject technology is to operate a laser device as an optical plasma drill, which involves (i) causing a plasma ball to form near the distal end of the optical waveguide of the laser device, (ii) terminating laser emission to extinguish the plasma ball and protect the fiber optic tip from excessive damage, and (iii) re-enabling emission to ignite another plasma ball-thus forming an ongoing sequence of transient plasma ball expansions and allowing the laser device to “drill” through tissue or other materials.

A continuing sequence of short-duration plasma balls may be applied or put in direct contact with tissue or materials in which a small hole is desired. Due to the high temperature of a plasma ball, virtually anything can be drilled or melted through. A flexible waveguide (e.g., silica fiber optic) also has the advantage of being easily positioned in hard to reach locations, such as deep in a human body. Optical waveguides may also be delivered via an endoscope or other type of scope via a working channel in the scope to an otherwise difficult to access location.

The amount of time for which laser output is disabled can be optimized in order to protect the optical waveguide from damage while also prioritizing drilling speed. In determining downtime, other factors can be considered, such as the amount of time needed for the plasma ball to extinguish, the amount of time needed for the waveguide to cool thereafter, and the amount of time needed for surrounding tissue to cool to avoid damage thereto. For example laser off time between plasma ball formations may be extended to reduce hemostasis and prevent ablation or thermal damage outside the directly exposed tissue areas, such as regions outside of the drilled hole.

Of particular note is the nature of plasma ball expansion and absorption of laser irradiation. Following formation of a plasma ball, the ball will tend to absorb the totality of incident laser irradiation, thus driving a rapid expansion of the plasma ball. In most tissues or materials, only a portion of incident laser irradiation is absorbed. By contrast, after a plasma ball forms, nearly all of the applied laser irradiation incident on the plasma ball will be absorbed by the ball. Accordingly, once a plasma ball forms, it thereafter rapidly expands driven by the ongoing and now fully absorbed laser emission.

Off time duration can be selected to account for a variety of factors, including characteristics of the laser device and lasing parameters for a particular surgical procedure. The duration can be determined, for instance, based on a target tissue type. For example, 455 nanometer laser irradiation is highly absorbed in hemoglobin. Thus, tissues with a high density of hemoglobin may use a fixed off-time duration that is relatively longer than laser off-time durations for tissue with relatively less hemoglobin density. So, for example, a hole drilled in a blood-rich organ (e.g., liver) may benefit by use of a longer off time between plasma balls, whereas less blood-rich tissue (e.g., cartilage) may allow for shorter off times between sequential plasma ball formation.

Additionally, various parameters discussed herein can be individually selected by a user. For instance, a user can indicate a shutdown threshold for detecting a plasma ball and turning of laser output. As another example, a user can select the amount of time for which the laser device turns off after detecting a plasma ball before reactivating its laser source.

It is noted that long-duration emission is the key to entering the thermal diffusion domain (e.g., for plasma ball formation); however, ultra-short nano- or pico-second duration emissions of sufficiently high peak power may also initiate plasma ignition or formation. Thus, long-duration emissions of as little as 10 watts or 15 kilowatts per centimeter squared in 100 milliseconds to continuous-wave durations on high-absorbing targets such as 455 nanometers and hemoglobin or soft tissues or conversely very high-peak irradiance in a very short pulse duration domain, such as nano- or pico-second duration, can both act to establish a plasma ball sufficient to cut, drill, or ablate almost any material or tissue.

There are many applications—surgical and otherwise—that benefit from the capability to drill or ablate small holes. For example, micro-fracturing where knee cartilage is rejuvenated by means of a matrix of small holes drilled through the cartilage and into the bone such that some bleeding occurs and enhanced perfusion of the cartilage is encouraged. In such procedures, use of an endoscope to deliver an optical waveguide may allow for a less invasive clinical approach. Additionally, the fiber optic may be delivered via a handpiece or the optical waveguide may even be held by hand directly. The fiber tip surface is placed directly on the targeted tissue or in close proximity thereof, thusly allowing the plasma ball to ablate or vaporize the targeted region.

Discussion of Drawings. Reference will now be made to implementations, examples of which are illustrated in the accompanying drawings. In the following description, specific details are set forth in order to provide an understanding of the various described implementations. However, it will be apparent to one of ordinary skill in the art that the various described implementations may be practiced without one or more of these details. In other instances, well-known methods, procedures, components, circuits, and networks have not been described in detail so as not to unnecessarily obscure aspects of the implementations.

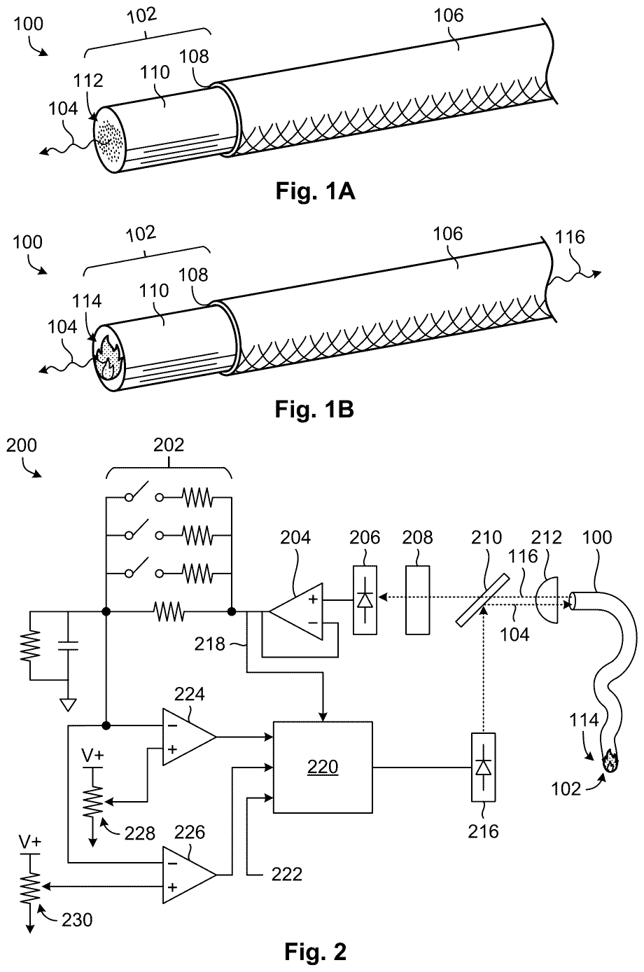

FIGS. 1A and 1B illustrate an example optical waveguide 100 of a laser device (e.g., laser device 200, below) configured to operate as an optical plasma drill, according to various aspects of the subject technology. The optical waveguide 100 includes an outer buffer layer 106, a cladding layer 108, and a core 110. In some embodiments, the optical waveguide 100 is a fiber optic cable, with the core 110 including one or more fiber optic strands. During standard operation, the optical waveguide 100 emits a laser beam 104 from the distal end 102 thereof.

As illustrated in FIG. 1A, contaminants 112 can accrue on the distal end 102 of the optical waveguide 100. For instance, during a surgical procedure, tissue or fluids can accrue on the end 102 of the waveguide 100. When such contaminants 112 are exposed to the beam 104, the contaminants 112 can ignite and form a plasma ball 114. This is illustrated in FIG. 1B.

The plasma ball 114 can be used to drill through tissue or other materials as discussed in more detail herein. However, the ball 114 can also damage the optical waveguide 100 if allowed to burn for too long. Accordingly, the optical waveguide 100 facilitates detection of the plasma ball 114 to assist the aforenoted laser device in determining when to stop emitting a laser and allow the plasma ball 114 to extinguish. This detection involves the optical waveguide transmitting light 116 emit by the plasma ball 114 towards a proximal end (not pictured) of the optical waveguide 100. This light 116 can be used to determine when a plasma ball has formed—for instance, based on whether a magnitude of the light 116 satisfies (e.g., meets, exceeds) a predetermined threshold (referred to herein as a “shutdown threshold”).

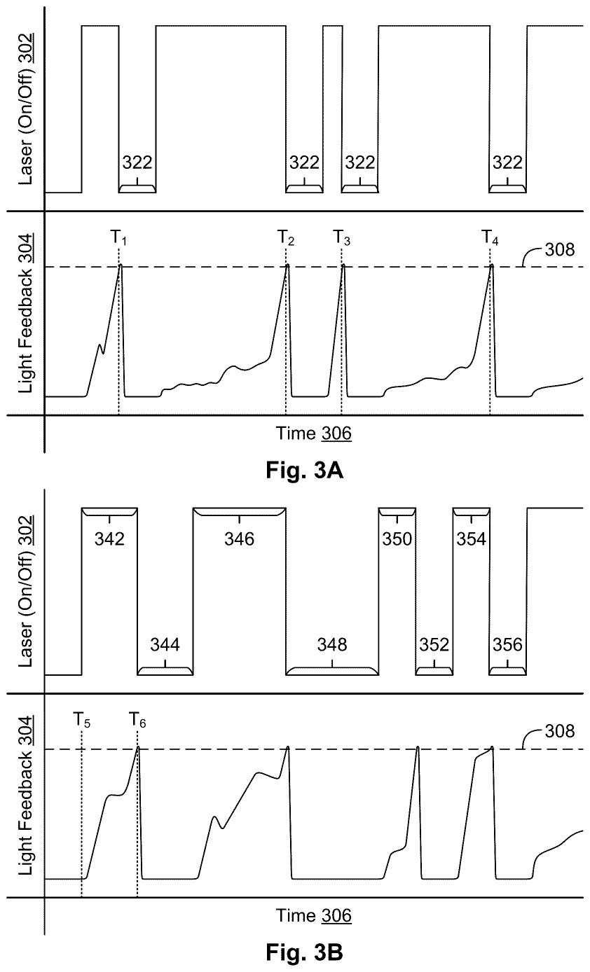

FIG. 2 illustrates example components of a laser device 200 configured to operate as an optical plasma drill, according to various aspects of the subject technology. The laser device 200 can repeatedly form plasma balls 114 at a distal end 102 of its optical waveguide 100 in order to drill holes in varying types of tissue, including cartilage, bone, soft tissue, or and the like.

The laser device 200 includes a laser source 216 configured to emit a laser beam 104. In the illustrated embodiment, the laser beam 104 is reflected by a turning mirror 210 through a focusing lens 212 and into an optical waveguide 100. After traveling the length of the optical waveguide 100, the laser beam 104 is emit at the distal end 102 thereof, whereat the laser beam 104 can cause the formation of a plasma ball 114 by igniting contamination (e.g., contaminants 112) on the waveguide 100. In some embodiments, the laser source 216 emits the laser beam 104 with pulse durations in excess of 30-50 milliseconds in order to encourage plasma ball 114 formation.

As discussed for FIG. 1B, broadband visible light emissions 116 from the plasma ball 114 propagate back up through the optical waveguide 100, thus enabling the laser device 200 to detect the plasma ball 114. This light 116 travels through the optical waveguide 100, through the turning mirror 210, and through an optical filter 208 configured to block radiation from the laser beam 104. After traveling through the optical filter 208, the light 116 travels to a photodetector 206 configured to generate a signal corresponding to an amount of the light 116, which amount can be compared against a shutdown threshold to determine whether to cause the laser source 216 to stop emitting the laser beam 104 (see integrated circuit 220, below).

The signal generated by the photodetector 206 is provided to a buffer amplifier 204, the output of which is delivered to an integrated circuit 220 (via path 218) and to an adjustable, user-selectable time-delay circuit 202. The integrated circuit 220 is configured to determine whether the amount of light 116 satisfies the shutdown threshold and an amount of time for disabling the laser source 216 responsive to the amount of light 116 satisfying said threshold. And the time-delay circuit 202 is configured to allow a user to introduce a delay between determining that the amount of light 116 satisfies the shutdown threshold and disabling the laser source 216.

The output of the time-delay circuit 202 is fed to a first comparator 224, with a user-adjustable threshold 228 (e.g., shutdown threshold 308, below) and where the output of the comparator 224 is delivered to the integrated circuit 220. Inputs to the integrated circuit 220 also include the output of a second comparator 226 tied to another user-adjustable threshold 230 (e.g., intermediary threshold 310, below). Other inputs to the integrated circuit 220 include user inputs 222. For instance, the user inputs 222 can indicate whether the integrated circuit 220 should operate in a fixed off-time mode, a proportionate off-time mode, or an integrated, area-under-the-curve off-time mode. The user inputs 222 can also indicate proportionality selections (e.g., a metric for scaling off-time determinations under any of the aforenoted modes).

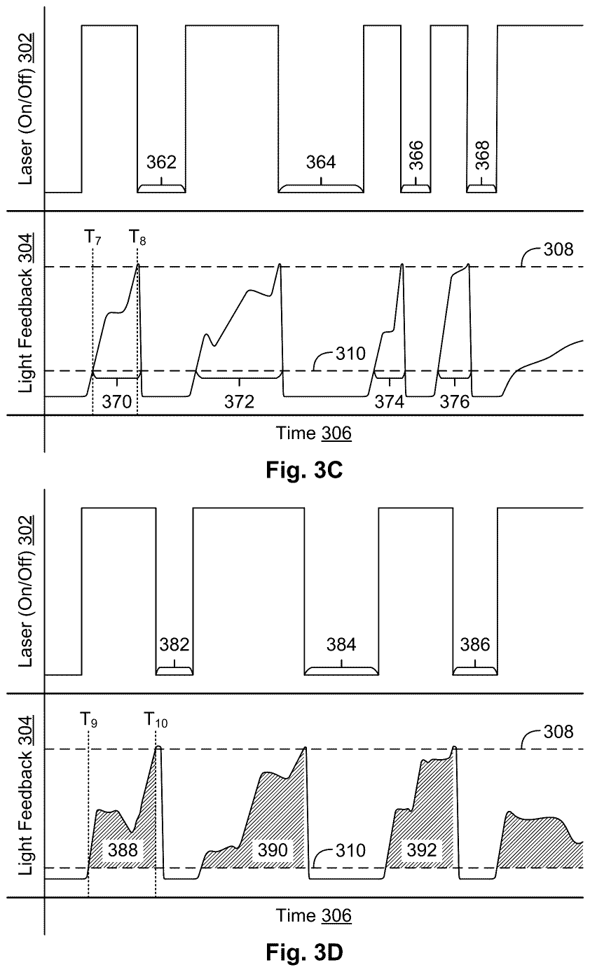

FIGS. 3A-3D illustrate example activity 302 of a laser device (e.g., laser device 200) operating as an optical plasma drill, according to various aspects of the subject technology. The laser device is configured to activate and deactivate its laser source (e.g., laser source 216) over time 306 based on light feedback 304 (e.g., light 116) detected by a photodetector (e.g., photodetector 206) of the laser device, which light can be compared to a shutdown threshold 308 to determine whether a plasma ball (e.g., plasma ball 114) has formed at an optical waveguide (e.g., optical waveguide 100) of the laser device.

After detecting that the amount of light 304 satisfies (e.g., meets, exceeds) the shutdown threshold 308, the laser device deactivates its laser source to allow the detected plasma ball to extinguish and prevent damage to the optical waveguide. For instance, in FIG. 3A, the laser device detects formation of a plasma ball at times T₁, T₂, T₃, and T₄. At each of these times, the laser device deactivates its laser source for the same amount of time, indicated as time 322. Such operation can be referred to as fixed off-time mode.

The time 322 for which the laser source is deactivated following detection of a plasma ball can be selected by a user (e.g., 100 milliseconds). In some embodiments, the fixed amount of time 322 can be determined based on a type of tissue targeted for drilling. For example, the user can provide an indication of the tissue type (e.g., user inputs 222) to the laser device and the laser device can determine the amount of time 322 by indexing a lookup table using the tissue type. In this manner, the amount of time 322 can be adjusted to account for the approximate thermal conductivity of a given target tissue such that thermally conductive or fluid-rich tissues are allowed a shorter off time and thermally resistive tissues (e.g., hard bone) are provided with a relatively longer duration off time to provide more time for fiber optic tip surface cooling.

In FIG. 3B, the off-time is proportionate to the on-time of the laser device. This operation can be referred to as proportionate off-time mode. While operating in this mode, the laser source emits a laser beam (e.g., laser beam 104) from time T₅ until time T₆, at which point the amount of light feedback 304 satisfies the shutdown threshold 308 and the laser device deactivates its laser source. Then, rather than deactivating the laser source for a fixed amount of time (as in FIG. 3A), the laser device deactivates the laser source for an amount of time proportionate to the amount of time for which the laser device was active.

For instance, after operating the laser source for a first amount of time 342 (i.e., from T₅ to T₆), the laser device deactivates the laser source for the same amount of time 344. Then, the laser device reactivates the laser source for a second amount of time 346 and deactivates it for the same amount of time 348 after detecting that the amount of light 304 again satisfies the shutdown threshold 308 (and so on for times 350 and 352, as well as times 354 and 356). It is noted that, although the on- and off-times in the illustrated embodiment are the same, the off-time need only be proportionate to the corresponding (e.g., immediately preceding) on-time while operating in proportionate off-time mode. For instance, the off-time can be equal to the on-time as multiplied by a predetermined constant (e.g., selected by a user).

In this example, the proportionality of on-time versus off-time durations remains fixed and equal to the previous on time; however, that proportionality may be also user-adjustable where, for example, the user can select the off time to be fixed at equal to one-half of the on-time or where the user may select the off time to equal to double or triple the on-time. Similar to the fixed off-time example in FIG. 3A, the user may be provided an input to adjust laser off-time proportionality to the preceding on-time duration in order to optimize or minimize laser off time while still providing optimized time for fiber optic tip surface cooling. Alternatively, the proportionality of the off-time and the on-time can be determined based on a user-provided input, such as a type of the tissue being targeted for lasing.

In FIG. 3C, the laser device uses an intermediary threshold 310 for further refining the determination regarding the amount of time for disabling the laser source. The intermediary threshold 310 can be set by a user or determined by the laser device, for instance, based on characteristics of the laser device or lasing parameters for a surgical procedure.

In FIG. 3C, the laser device determines the amount of time for disabling the laser source based on an amount of time between the amount of light 304 satisfying the intermediary threshold (e.g., at T₇) and satisfying the shutdown threshold (e.g., at T₈). For example, after the amount of light exceeds the threshold for a first amount of time 370, the laser source is deactivated for an amount of time 362 proportionate to the first amount of time. This continues for a second amount of time 372 and a corresponding off time 364, a third amount of time 374 and a corresponding off time 366, and a fourth amount of time 376 and a corresponding off time 368.

The final illustrated mode of FIG. 3D is referred to herein as integrated, area-under-the-curve off-time mode (or simply integrated mode). Here, the amount of light 304 satisfies the shutdown threshold 308 at time T₁₀, after which the laser source is disabled for an amount of time 382 proportionate to the area 388 under the curve of the amount of light 304 as a function of time 306. In the illustrated embodiment, the area 388 is further limited by the aforenoted intermediary threshold 388—though, in some embodiments, the entire area under the curve is used for determining the amount of time for disabling the laser source (e.g., intermediary threshold 310 is zero).

In general, the heat aggregated at the distal end of the optical waveguide will be approximately equal to the integrated intensity of emission from ignited material over the duration between the ignition on and ignition limit thresholds, thereby providing a reference to determine the minimum off time or cooling time for the fiber optic surface and thusly maximizing ignition on time to facilitate optimal fiber tip ignition cutting power. In particular, where tissues of varying composition and therefore varying thermal conductivity the implementation of an area under the curve off time generator provide optimal functionality to adequately cool the fiber optic tip surface while maintaining maximized laser on time and thus maximized fiber tip surface proximal ignition or ignited plasma for maximized cutting or drilling capability.

In some embodiments, if the detected light 304 satisfies the intermediary threshold 310 for a predetermined amount of time, the laser emission may be terminated even if the light 304 does not satisfy the upper shutdown threshold 308. This option is available for each of the operating modes described above (e.g., fixed, proportional, and integrated modes) in order to better protect the optical waveguide from heat damage. In such embodiments, in integrated mode, where the intermediary threshold 310 is satisfied but the shutdown threshold 308 is not, the laser emission may be terminated after the area under the curve and above the intermediary threshold 310 satisfies an area threshold (e.g., selected by the user). In this manner, very long duration of lasing above intermediary 310 but below shutdown threshold 308 may be appended to protect the fiber tip.

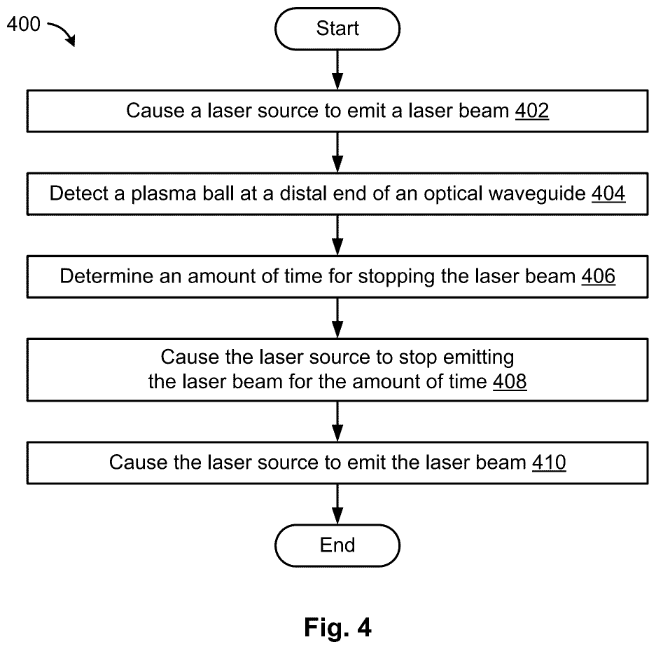

FIG. 4 illustrates an example process 400 for operating a laser device as an optical plasma drill, according to various aspects of the subject technology. The operations of the process 400 can be executed by devices discussed above with respect to FIGS. 1-3D, such as the laser device 200 of FIG. 2.

In some embodiments, instructions stored in a non-transitory, computer-readable medium correspond to operations of the example process 400. In such embodiments, a processor of an electronic device (e.g., laser device 200) can execute the instructions to cause the electronic device to perform the corresponding operations. It is noted that some or all of the circuitry described herein (e.g., for FIG. 2) can be replaced by a processor, memory, and/or corresponding instructions. For example, the comparators 224 and 226 and threshold circuitry 228 and 230 of FIG. 2 can be replaced with instructions for determining whether certain values satisfy predetermined thresholds (e.g., shutdown threshold 308, intermediary threshold 310).

The process 400 includes causing (402) a laser source (e.g., laser source 216) to emit a laser beam. The process 400 also includes detecting (404) a plasma ball (e.g., plasma ball 114) at a distal end (e.g., distal end 102) of an optical waveguide (e.g., optical waveguide 100). For example, detecting the plasma ball can include detecting an amount of light (e.g., light 234, light feedback 304) emit at the proximal end of the optical waveguide and determining that the amount of light satisfies a shutdown threshold (e.g., threshold 308). The light can be received at the distal end of the optical waveguide and caused by ignition of contamination (e.g., contamination 112) located at the distal end of the optical waveguide.

Additionally, the process 400 includes determining (406) an amount of time (e.g., time 344, time 362, time 382) for stopping the laser beam. Rather than relying on a static, pre-programmed amount of time (as in FIG. 3A), various metrics can be used to better determine how long the laser beam should be deactivated. For instance, the amount of time can be determined based on an amount of time for which the laser beam was active prior to detecting the plasma ball (see FIG. 3B), based on an amount of time for which the amount of light exceeds an intermediary threshold (e.g., threshold 310) prior to detecting the plasma ball (see FIG. 3C), or based on an area (e.g., area 388) between the intermediary threshold and the amount of light (e.g., light feedback 304) as a function of time (e.g., time 306) (see FIG. 3D).

Further, the process 400 includes causing (408) the laser source to stop emitting the laser beam for the amount of time and causing (410) the laser source to emit the laser beam after the amount of time. Stopping the laser source from emitting the laser beam allows the plasma ball to extinguish and helps prevent damage to the optical waveguide. Causing the laser source to again emit the laser beam leads to formation of another plasma ball, which causes target tissue to break down and allows the laser device to operate as an optical plasma drill.

In some embodiments, the process 400 further includes determining an amount of time (e.g., time 342) between causing the laser source to emit the laser beam (e.g., at T₅) and detecting the plasma ball (e.g., at T₆). In such embodiments, determining the amount of time (e.g., time 344) for stopping the laser beam may be based on (e.g., equal to, proportionate to) the amount of time between causing the laser source to emit the laser beam and detecting the laser ball.

In some embodiments, the process 400 further includes determining an intermediary threshold (e.g., intermediary threshold 310) based on laser characteristics or lasing parameters for a surgical procedure, where the intermediary threshold is less than the shutdown threshold. In such embodiments, determining the amount of time for stopping the laser beam can be based on the intermediary threshold and the amount of light. Also, in such embodiments, the laser characteristics can include an inner diameter of the optical waveguide, an outer diameter of the optical waveguide, or an amount of noise expected when detecting the amount of light. Further, in such embodiments, the lasing parameters can include an indication of whether the optical waveguide will be in an open-air environment or a saline environment during the surgical procedure, an indication of whether the surgical procedure will involve a doping material (e.g., a dye or other chromophore for absorbing laser emission and encouraging plasma formation, or a liquid for cooling the optical waveguide), or an indication of a tissue type for lasing during the surgical procedure.

In some embodiments, the process 400 further includes determining an amount of time (e.g., time 370) between the amount of light (e.g., light feedback 304) satisfying the intermediary threshold (e.g., at T₇) and the amount of light satisfying the shutdown threshold (e.g., at T₈). In such embodiments, determining the amount of time (e.g., time 362) for stopping the laser beam can be based on (e.g., equal to, proportionate to) the amount of time between the amount of light satisfying the intermediary threshold and the amount of light satisfying the shutdown threshold.

In some embodiments, the process 400 further includes determining an area (e.g., area 388) between the intermediary threshold and the amount of light (e.g., light feedback 304) as a function of time (e.g., time 306), bounded by the amount of light satisfying the intermediary threshold (e.g., at T₉) and the amount of light satisfying the shutdown threshold (e.g., at T₁₀). In such embodiments, determining the amount of time (e.g., time 382) for stopping the laser beam can be based on (e.g., proportionate to) the area between the intermediary threshold and the amount of light as a function of time, bounded by the amount of light satisfying the intermediary threshold and the amount of light satisfying the shutdown threshold.

In some embodiments, the process 400 further includes detecting a shutdown threshold (e.g., shutdown threshold 308) and an intermediary threshold (e.g., intermediary threshold 310) less than the shutdown threshold. In such embodiments, the shutdown and intermediary thresholds can be established by threshold circuitry (e.g., threshold circuitry 82 or 87). Additionally, in such embodiments, the process 400 can further include detecting via a photodetector an amount of light emit at the proximal end of the optical waveguide. Also, in such embodiments, the light can be received at the distal end of the optical waveguide and caused by ignition of contamination (e.g., contamination 112) located at the distal end of the optical waveguide. Further, in such embodiments, detecting the plasma ball can include determining that the amount of light satisfies the shutdown threshold. Moreover, in such embodiments, the process 400 can include calculating via an integrator (e.g., integrated circuit 220) an area (e.g., area 388) between the intermediary threshold and the amount of light (e.g., light feedback 304) as a function of time (e.g., time 306), bounded by the amount of light satisfying the intermediary threshold (e.g., at T₉) and the amount of light satisfying the shutdown threshold (e.g., at T₁₀). Furthermore, in such embodiments, determining the amount of time for stopping the laser beam can be based on (e.g., proportionate to) the area between the intermediary threshold and the amount of light as a function of time, bounded by the amount of light satisfying the intermediary threshold and the amount of light satisfying the shutdown threshold.

In some embodiments, the laser beam includes monochromatic light with a power output of approximately 30 watts (e.g., ±10 watts) and a wavelength of approximately 455 nanometers (e.g., ±50 nanometers).

Additional Embodiments. A preferred embodiment includes the use of a silica-fiber-coupled 455 nanometer diode laser capable of continuous-wave operation, as well as long or short pulses and variations thereof. The embodiment also includes a broadband light-detection apparatus configured to receive visible emissions from ignited or plasma radiation proximal to or on the fiber tip surface, where said apparatus detects the magnitude and time duration of visible ignition emissions and which includes time-delaying circuitry, as well as adjustable user thresholds for ignition limit and ignition on detecting comparators-all of which is fed to a laser off-time generator used to determine the cooling period between re-ignited emissions and which inhibits laser emissions when the ignited emissions exceed the ignition limit threshold. In some embodiments, the off-time generator provides multiple modes of off-time determination, including, for instance, fixed mode, proportional mode, and integrated mode.

The preferred off-time selection mode for most surgical applications in tissues of variable composition is the integrated, area-under-the-curve mode. This mode may also include a proportionality constant which can be user-selectable and which can act to multiply the integrated off-time duration by a constant to either increase or decrease the resulting off time in proportion to that which is indicated to be at unity with the integrated photodetector area-under-the-curve between the ignition-on and ignition-off thresholds, where the time begins to accumulate after the photodiode signal crosses above the on-time threshold and either rises to the ignition limit threshold, thus ending the integrated time period or until the photodiode signal falls back below the ignition on threshold. This ends the integrated time period and results in a laser-inhibition or laser-off time period proportional to the energy measured by the photodiode.

Conversely, the user may prefer using either the proportional off-time mode to fine tune optical fiber tip wear to an acceptable level when operating in single-tissue type regions, such as through cartilage, or bone, or other tissue. In this case, a user-selected proportionality constant would multiply by the off-time derived by measuring the previous on-time period where the photodiode signal crosses above the ignition-on threshold and then either falls back below this threshold or continues on until it crosses the upper-ignition-limit threshold to generate a user-selected, proportionately compensated off-time based on the original proportional on-time above the ignition threshold. This allows for either increasing or decreasing the fiber optic tip wear.

After a plasma ball ignition, the subsequent detection thereof, and resulting laser emission cessation, the laser-inhibit or off-time prior to automatic re-initiation of laser emission may be for a preselected, fixed time period based on the lasing environment (e.g., water, saline, air) or based on the tissue or material type. A fixed, laser-off time interval may even be based on laser power where lower power settings allow for a shorter duration off-time and where high power laser emission results in a longer laser-off time. Likewise, the laser-off time may be a variable based on the plasma broadband light emissions rate of rise such that rapidly expanding plasma light emissions may result in a shorter laser-off time duration and where slower rising plasma light emissions allow for a longer off time before re-initiation of laser emission.

The addition of externally applied plasma ignition contamination is preferred-such as blood, water, dye, and the like. For example, 455 nanometer lasers are highly absorbed by hemoglobin or even red dyes and where carbon-dioxide lasers are highly absorbed by water or saline. The 455 nanometer ignition may be stimulated by addition of blood, hemoglobin, red dyes or other material whether liquid, powder or solid whereas the carbon-dioxide ignition may be stimulated by the addition of water, saline, other dyes, or the like. In the case of the carbon-dioxide, a suitably transparent to 10,600 nanometer wavelength waveguide may be used to deliver the laser emission to the target tissue area. In this case, the distal end of the optical waveguide in contact with the target may be of virtually any shape (e.g., beyond just circular) such that drilling pre-shaped channels in the shape of the waveguide distal surface is possible. Such channels may be helpful for orthopedic-surgeon installed components (e.g., metal pins, anchors) to be pre-fit to shapes other than circular, thereby facilitating implantation of various mechanical devices into bones. Additionally Erbium wavelengths such as 2,940 nanometers or other wavelengths up to around 3,500 nanometers or so wavelengths may be suitable for shaped waveguides for use in ablating pre-shaped channels in bones or related tissues also to facilitate implantation of anchors, metal pieces, and the like. Circular waveguides for 2,940-3,500 nanometer wavelengths are also envisioned, as well as typical cylindrical or circular fiber optics.

Total extinguishing of a plasma ball is preferred but is not absolutely required. For some applications, it may be beneficial to simply reduce plasma magnitude by greatly reducing laser power such that the plasma nearly extinguishes. Thereafter laser power can be increased to re-expand the plasma ball. This may also be acceptable in cases of highly thermally conductive environments, such as saline, water, or other fluid-infused areas. This may also be useful where the type of contamination present on the fiber optic tip is difficult to re-ignite. Thus, a greatly reduced plasma ball may function as a seed for subsequent laser emission, resulting in a rapid, reliable plasma re-ignition. In this case short bursts of sequential plasma ignitions may allow for brief drilling periods (such as during the burst of sequential laser pulses) and where the laser is inhibited to enforce a cooling period between that of a laser pulse burst set. Thus, optimal drilling laser pulse bursts followed by brief, adjustable inter-pulse cooling periods provide limitation of fiber silica temperatures.

Illustrative Clauses. For further reference, example aspects of the present disclosure are included below as numbered clauses. These clauses are provided for illustrative purposes and are not intended to limit the subject technology.

Clause 1. A laser device comprising: a laser source configured to emit a laser beam; an optical waveguide comprising proximal and distal ends, wherein the optical waveguide is positioned to receive the laser beam from the laser source at the proximal end and emit the laser beam at the distal end; and control circuitry configured to: cause the laser source to emit the laser beam; detect a plasma ball at the distal end of the optical waveguide; responsive to detecting the plasma ball, (i) determine an amount of time for stopping the laser beam and (ii) cause the laser source to stop emitting the laser beam for the amount of time; and cause the laser source to emit the laser beam after the amount of time.

Clause 2. The laser device of Clause 1, wherein: the control circuitry is further configured to determine an amount of time between causing the laser source to emit the laser beam and detecting the plasma ball; and determining the amount of time for stopping the laser beam is based on the amount of time between causing the laser source to emit the laser beam and detecting the laser ball.

Clause 3. The laser device of either Clause 1 or 2, wherein detecting the plasma ball comprises: detecting an amount of light emit at the proximal end of the optical waveguide, wherein the light is received at the distal end of the optical waveguide and caused by ignition of contamination located at the distal end of the optical waveguide; and determining that the amount of light satisfies a shutdown threshold.

Clause 4. The laser device of Clause 3, wherein: the control circuitry is further configured to determine an intermediary threshold based on laser characteristics or lasing parameters for a surgical procedure, wherein the intermediary threshold is less than the shutdown threshold; and determining the amount of time for stopping the laser beam is based on the intermediary threshold and the amount of light.

Clause 5. The laser device of Clause 4, wherein the laser characteristics comprise two or more of (i) an inner diameter of the optical waveguide, (ii) an outer diameter of the optical waveguide, and (iii) an amount of noise expected when detecting the amount of light.

Clause 6. The laser device of either Clause 4 or 5, wherein the lasing parameters comprise two or more of (i) an indication of whether the optical waveguide will be in an open-air environment or a saline environment during the surgical procedure, (ii) an indication of whether the surgical procedure will involve a doping material, and (iii) an indication of a tissue type for lasing during the surgical procedure.

Clause 7. The laser device of any one of Clauses 4-6, wherein: the control circuitry is further configured to determine an amount of time between the amount of light satisfying the intermediary threshold and the amount of light satisfying the shutdown threshold; and determining the amount of time for stopping the laser beam is based on the amount of time between the amount of light satisfying the intermediary threshold and the amount of light satisfying the shutdown threshold.

Clause 8. The laser device of any one of Clauses 4-7, wherein: the control circuitry is further configured to determine an area between the intermediary threshold and the amount of light as a function of time, bounded by the amount of light satisfying the intermediary threshold and the amount of light satisfying the shutdown threshold; and determining the amount of time for stopping the laser beam is based on the area between the intermediary threshold and the amount of light as a function of time, bounded by the amount of light satisfying the intermediary threshold and the amount of light satisfying the shutdown threshold.

Clause 9. The laser device of any one of Clauses 1-8, wherein the control circuitry further comprises: threshold circuitry configured to establish (i) a shutdown threshold and (ii) an intermediary threshold less than the shutdown threshold; a photodetector configured to detect an amount of light emit at the proximal end of the optical waveguide, wherein the light is received at the distal end of the optical waveguide and caused by ignition of contamination located at the distal end of the optical waveguide, and detecting the plasma ball comprises determining that the amount of light satisfies the shutdown threshold; and an integrator configured to calculate an area between the intermediary threshold and the amount of light as a function of time, bounded by the amount of light satisfying the intermediary threshold and the amount of light satisfying the shutdown threshold, wherein determining the amount of time for stopping the laser beam is based on the area between the intermediary threshold and the amount of light as a function of time, bounded by the amount of light satisfying the intermediary threshold and the amount of light satisfying the shutdown threshold.

Clause 10. The laser device of any one of Clauses 1-9, wherein the laser beam comprises monochromatic light with a power output of approximately 30 watts and a wavelength of approximately 455 nanometers.

Clause 11. A non-transitory, computer-readable medium storing instructions that, when executed by a processor of an electronic device, cause the electronic device to perform operations comprising: causing a laser source to emit a laser beam; detecting a plasma ball at a distal end of an optical waveguide, wherein the optical waveguide comprises a proximal end and the distal end, and the optical waveguide is positioned to receive the laser beam from the laser source at the proximal end and emit the laser beam at the distal end; responsive to detecting the plasma ball, (i) determining an amount of time for stopping the laser beam and (ii) causing the laser source to stop emitting the laser beam for the amount of time; and causing the laser source to emit the laser beam after the amount of time.

Clause 12. The non-transitory, computer-readable medium of Clause 11, wherein the operations further comprise: determining an amount of time between causing the laser source to emit the laser beam and detecting the plasma ball; wherein determining the amount of time for stopping the laser beam is based on the amount of time between causing the laser source to emit the laser beam and detecting the laser ball.

Clause 13. The non-transitory, computer-readable medium of either Clause 11 or 12, wherein detecting the plasma ball comprises: detecting an amount of light emit at the proximal end of the optical waveguide, wherein the light is received at the distal end of the optical waveguide and caused by ignition of contamination located at the distal end of the optical waveguide; and determining that the amount of light satisfies a shutdown threshold.

Clause 14. The non-transitory, computer-readable medium of Clause 13, wherein the operations further comprise: determining an intermediary threshold based on laser characteristics or lasing parameters for a surgical procedure, wherein the intermediary threshold is less than the shutdown threshold; wherein determining the amount of time for stopping the laser beam is based on the intermediary threshold and the amount of light.

Clause 15. The non-transitory, computer-readable medium of Clause 14, wherein the laser characteristics comprise two or more of (i) an inner diameter of the optical waveguide, (ii) an outer diameter of the optical waveguide, and (iii) an amount of noise expected when detecting the amount of light.

Clause 16. The non-transitory, computer-readable medium of either Clause 14 or 15, wherein the lasing parameters comprise two or more of (i) an indication of whether the optical waveguide will be in an open-air environment or a saline environment during the surgical procedure, (ii) an indication of whether the surgical procedure will involve a doping material, and (iii) an indication of a tissue type for lasing during the surgical procedure.

Clause 17. The non-transitory, computer-readable medium of any one of Clauses 14-16, wherein the operations further comprise: determining an amount of time between the amount of light satisfying the intermediary threshold and the amount of light satisfying the shutdown threshold; wherein determining the amount of time for stopping the laser beam is based on the amount of time between the amount of light satisfying the intermediary threshold and the amount of light satisfying the shutdown threshold.

Clause 18. The non-transitory, computer-readable medium of any one of Clauses 14-17, wherein the operations further comprise: determining an area between the intermediary threshold and the amount of light as a function of time, bounded by the amount of light satisfying the intermediary threshold and the amount of light satisfying the shutdown threshold; wherein determining the amount of time for stopping the laser beam is based on the area between the intermediary threshold and the amount of light as a function of time, bounded by the amount of light satisfying the intermediary threshold and the amount of light satisfying the shutdown threshold.

Clause 19. The non-transitory, computer-readable medium of any one of Clauses 11-18, wherein the operations further comprise: detecting (i) a shutdown threshold and (ii) an intermediary threshold less than the shutdown threshold, wherein the shutdown and intermediary thresholds are established by threshold circuitry; detecting via a photodetector an amount of light emit at the proximal end of the optical waveguide, wherein the light is received at the distal end of the optical waveguide and caused by ignition of contamination located at the distal end of the optical waveguide, and detecting the plasma ball comprises determining that the amount of light satisfies the shutdown threshold; and calculating via an integrator an area between the intermediary threshold and the amount of light as a function of time, bounded by the amount of light satisfying the intermediary threshold and the amount of light satisfying the shutdown threshold, wherein determining the amount of time for stopping the laser beam is based on the area between the intermediary threshold and the amount of light as a function of time, bounded by the amount of light satisfying the intermediary threshold and the amount of light satisfying the shutdown threshold.

Clause 20. The non-transitory, computer-readable medium of any one of Clauses 11-19, wherein the laser beam comprises monochromatic light with a power output of approximately 30 watts and a wavelength of approximately 455 nanometers.

Clause 21. A computer-implemented method for operating a laser device as an optical plasma drill, the method comprising: causing a laser source to emit a laser beam; detecting a plasma ball at a distal end of an optical waveguide, wherein the optical waveguide comprises a proximal end and the distal end, and the optical waveguide is positioned to receive the laser beam from the laser source at the proximal end and emit the laser beam at the distal end; responsive to detecting the plasma ball, (i) determining an amount of time for stopping the laser beam and (ii) causing the laser source to stop emitting the laser beam for the amount of time; and causing the laser source to emit the laser beam after the amount of time.

Clause 22. The computer-implemented method of Clause 21, further comprising: determining an amount of time between causing the laser source to emit the laser beam and detecting the plasma ball; wherein determining the amount of time for stopping the laser beam is based on the amount of time between causing the laser source to emit the laser beam and detecting the laser ball.

Clause 23. The computer-implemented method of either Clause 21 or 22, wherein detecting the plasma ball comprises: detecting an amount of light emit at the proximal end of the optical waveguide, wherein the light is received at the distal end of the optical waveguide and caused by ignition of contamination located at the distal end of the optical waveguide; and determining that the amount of light satisfies a shutdown threshold.

Clause 24. The computer-implemented method of Clause 23, further comprising: determining an intermediary threshold based on laser characteristics or lasing parameters for a surgical procedure, wherein the intermediary threshold is less than the shutdown threshold; wherein determining the amount of time for stopping the laser beam is based on the intermediary threshold and the amount of light.

Clause 25. The computer-implemented method of Clause 24, wherein the laser characteristics comprise two or more of (i) an inner diameter of the optical waveguide, (ii) an outer diameter of the optical waveguide, and (iii) an amount of noise expected when detecting the amount of light.

Clause 26. The computer-implemented method of either Clause 24 or 25, wherein the lasing parameters comprise two or more of (i) an indication of whether the optical waveguide will be in an open-air environment or a saline environment during the surgical procedure, (ii) an indication of whether the surgical procedure will involve a doping material, and (iii) an indication of a tissue type for lasing during the surgical procedure.

Clause 27. The computer-implemented method of any one of Clauses 24-26, further comprising: determining an amount of time between the amount of light satisfying the intermediary threshold and the amount of light satisfying the shutdown threshold; wherein determining the amount of time for stopping the laser beam is based on the amount of time between the amount of light satisfying the intermediary threshold and the amount of light satisfying the shutdown threshold.

Clause 28. The computer-implemented method of any one of Clauses 24-27, further comprising: determining an area between the intermediary threshold and the amount of light as a function of time, bounded by the amount of light satisfying the intermediary threshold and the amount of light satisfying the shutdown threshold; wherein determining the amount of time for stopping the laser beam is based on the area between the intermediary threshold and the amount of light as a function of time, bounded by the amount of light satisfying the intermediary threshold and the amount of light satisfying the shutdown threshold.

Clause 29. The computer-implemented method of any one of Clauses 21-28, further comprising: detecting (i) a shutdown threshold and (ii) an intermediary threshold less than the shutdown threshold, wherein the shutdown and intermediary thresholds are established by threshold circuitry; detecting via a photodetector an amount of light emit at the proximal end of the optical waveguide, wherein the light is received at the distal end of the optical waveguide and caused by ignition of contamination located at the distal end of the optical waveguide, and detecting the plasma ball comprises determining that the amount of light satisfies the shutdown threshold; and calculating via an integrator an area between the intermediary threshold and the amount of light as a function of time, bounded by the amount of light satisfying the intermediary threshold and the amount of light satisfying the shutdown threshold, wherein determining the amount of time for stopping the laser beam is based on the area between the intermediary threshold and the amount of light as a function of time, bounded by the amount of light satisfying the intermediary threshold and the amount of light satisfying the shutdown threshold.

Clause 30. The computer-implemented method of any one of Clauses 21-29, wherein the laser beam comprises monochromatic light with a power output of approximately 30 watts and a wavelength of approximately 455 nanometers.

Clause 31. A method of manufacturing a laser device comprising: providing a laser source configured to emit a laser beam; providing an optical waveguide comprising proximal and distal ends; positioning the optical waveguide to receive the laser beam from the laser source at the proximal end and emit the laser beam at the distal end; providing control circuitry configured to: cause the laser source to emit the laser beam; detect a plasma ball at the distal end of the optical waveguide; responsive to detecting the plasma ball, (i) determine an amount of time for stopping the laser beam and (ii) cause the laser source to stop emitting the laser beam for the amount of time; and cause the laser source to emit the laser beam after the amount of time; and connecting the control circuitry to the laser source such that the control circuitry can cause the laser source to emit the laser beam.

Clause 32. The method of Clause 31, wherein: the control circuitry is further configured to determine an amount of time between causing the laser source to emit the laser beam and detecting the plasma ball; and determining the amount of time for stopping the laser beam is based on the amount of time between causing the laser source to emit the laser beam and detecting the laser ball.

Clause 33. The method of either Clause 31 or 32, wherein detecting the plasma ball comprises: detecting an amount of light emit at the proximal end of the optical waveguide, wherein the light is received at the distal end of the optical waveguide and caused by ignition of contamination located at the distal end of the optical waveguide; and determining that the amount of light satisfies a shutdown threshold.

Clause 34. The method of Clause 33, wherein: the control circuitry is further configured to determine an intermediary threshold based on laser characteristics or lasing parameters for a surgical procedure, wherein the intermediary threshold is less than the shutdown threshold; and determining the amount of time for stopping the laser beam is based on the intermediary threshold and the amount of light.

Clause 35. The method of Clause 34, wherein the laser characteristics comprise two or more of (i) an inner diameter of the optical waveguide, (ii) an outer diameter of the optical waveguide, and (iii) an amount of noise expected when detecting the amount of light.

Clause 36. The method of either Clause 34 or 35, wherein the lasing parameters comprise two or more of (i) an indication of whether the optical waveguide will be in an open-air environment or a saline environment during the surgical procedure, (ii) an indication of whether the surgical procedure will involve a doping material, and (iii) an indication of a tissue type for lasing during the surgical procedure.

Clause 37. The method of Clauses 34-36, wherein: the control circuitry is further configured to determine an amount of time between the amount of light satisfying the intermediary threshold and the amount of light satisfying the shutdown threshold; and determining the amount of time for stopping the laser beam is based on the amount of time between the amount of light satisfying the intermediary threshold and the amount of light satisfying the shutdown threshold.

Clause 38. The method of any one of Clauses 34-37, wherein: the control circuitry is further configured to determine an area between the intermediary threshold and the amount of light as a function of time, bounded by the amount of light satisfying the intermediary threshold and the amount of light satisfying the shutdown threshold; and determining the amount of time for stopping the laser beam is based on the area between the intermediary threshold and the amount of light as a function of time, bounded by the amount of light satisfying the intermediary threshold and the amount of light satisfying the shutdown threshold.

Clause 39. The method of any one of Clauses 31-38, further comprising: providing threshold circuitry configured to establish (i) a shutdown threshold and (ii) an intermediary threshold less than the shutdown threshold; providing a photodetector configured to detect an amount of light emit at the proximal end of the optical waveguide, wherein the light is received at the distal end of the optical waveguide and caused by ignition of contamination located at the distal end of the optical waveguide, and detecting the plasma ball comprises determining that the amount of light satisfies the shutdown threshold; and providing an integrator configured to calculate an area between the intermediary threshold and the amount of light as a function of time, bounded by the amount of light satisfying the intermediary threshold and the amount of light satisfying the shutdown threshold, wherein determining the amount of time for stopping the laser beam is based on the area between the intermediary threshold and the amount of light as a function of time, bounded by the amount of light satisfying the intermediary threshold and the amount of light satisfying the shutdown threshold.

Clause 40. The method of any one of Clauses 31-39, wherein the laser beam comprises monochromatic light with a power output of approximately 30 watts and a wavelength of approximately 455 nanometers.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims pre-sent elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not in-tended to be limited to the aspects shown herein, but is to be accorded the full scope consistent with the language claims, wherein reference to an element in the singular is not intend-ed to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined in-puts, preconfigured inputs, or user-initiated inputs for making the determination. In some embodiments, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine-learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A laser device comprising:
a laser source configured to emit a laser beam;
an optical waveguide comprising proximal and distal ends, wherein the optical waveguide is positioned to receive the laser beam from the laser source at the proximal end and emit the laser beam at the distal end; and
control circuitry configured to:
cause the laser source to emit the laser beam;
detect a plasma ball at the distal end of the optical waveguide;
responsive to detecting the plasma ball, (i) determine an amount of time for stopping the laser beam and (ii) cause the laser source to stop emitting the laser beam for the amount of time; and
cause the laser source to emit the laser beam after the amount of time.

2. The laser device of claim 1, wherein:
the control circuitry is further configured to determine an amount of time between causing the laser source to emit the laser beam and detecting the plasma ball; and
determining the amount of time for stopping the laser beam is based on the amount of time between causing the laser source to emit the laser beam and detecting the laser ball.

3. The laser device of claim 1, wherein detecting the plasma ball comprises:
detecting an amount of light emit at the proximal end of the optical waveguide, wherein the light is received at the distal end of the optical waveguide and caused by ignition of contamination located at the distal end of the optical waveguide; and
determining that the amount of light satisfies a shutdown threshold.

4. The laser device of claim 3, wherein:
the control circuitry is further configured to determine an intermediary threshold based on laser characteristics or lasing parameters for a surgical procedure, wherein the intermediary threshold is less than the shutdown threshold; and
determining the amount of time for stopping the laser beam is based on the intermediary threshold and the amount of light.

5. The laser device of claim 4, wherein the laser characteristics comprise two or more of (i) an inner diameter of the optical waveguide, (ii) an outer diameter of the optical waveguide, and (iii) an amount of noise expected when detecting the amount of light.

6. The laser device of claim 4, wherein the lasing parameters comprise two or more of (i) an indication of whether the optical waveguide will be in an open-air environment or a saline environment during the surgical procedure, (ii) an indication of whether the surgical procedure will involve a doping material, and (iii) an indication of a tissue type for lasing during the surgical procedure.

7. The laser device of claim 4, wherein:
the control circuitry is further configured to determine an amount of time between the amount of light satisfying the intermediary threshold and the amount of light satisfying the shutdown threshold; and
determining the amount of time for stopping the laser beam is based on the amount of time between the amount of light satisfying the intermediary threshold and the amount of light satisfying the shutdown threshold.

8. The laser device of claim 4, wherein:
the control circuitry is further configured to determine an area between the intermediary threshold and the amount of light as a function of time, bounded by the amount of light satisfying the intermediary threshold and the amount of light satisfying the shutdown threshold; and
determining the amount of time for stopping the laser beam is based on the area between the intermediary threshold and the amount of light as a function of time, bounded by the amount of light satisfying the intermediary threshold and the amount of light satisfying the shutdown threshold.

9. The laser device of claim 1, wherein the control circuitry further comprises:
threshold circuitry configured to establish (i) a shutdown threshold and (ii) an intermediary threshold less than the shutdown threshold;
a photodetector configured to detect an amount of light emit at the proximal end of the optical waveguide, wherein the light is received at the distal end of the optical waveguide and caused by ignition of contamination located at the distal end of the optical waveguide, and detecting the plasma ball comprises determining that the amount of light satisfies the shutdown threshold; and
an integrator configured to calculate an area between the intermediary threshold and the amount of light as a function of time, bounded by the amount of light satisfying the intermediary threshold and the amount of light satisfying the shutdown threshold, wherein determining the amount of time for stopping the laser beam is based on the area between the intermediary threshold and the amount of light as a function of time, bounded by the amount of light satisfying the intermediary threshold and the amount of light satisfying the shutdown threshold.

10. The laser device of claim 1, wherein the laser beam comprises monochromatic light with a power output of approximately 30 watts and a wavelength of approximately 455 nanometers.

11. A non-transitory, computer-readable medium storing instructions that, when executed by a processor of an electronic device, cause the electronic device to perform operations comprising:
causing a laser source to emit a laser beam;
detecting a plasma ball at a distal end of an optical waveguide, wherein the optical waveguide comprises a proximal end and the distal end, and the optical waveguide is positioned to receive the laser beam from the laser source at the proximal end and emit the laser beam at the distal end;
responsive to detecting the plasma ball, (i) determining an amount of time for stopping the laser beam and (ii) causing the laser source to stop emitting the laser beam for the amount of time; and
causing the laser source to emit the laser beam after the amount of time.

12. The non-transitory, computer-readable medium of claim 11, wherein the operations further comprise:
determining an amount of time between causing the laser source to emit the laser beam and detecting the plasma ball;
wherein determining the amount of time for stopping the laser beam is based on the amount of time between causing the laser source to emit the laser beam and detecting the laser ball.

13. The non-transitory, computer-readable medium of claim 11, wherein detecting the plasma ball comprises:
detecting an amount of light emit at the proximal end of the optical waveguide, wherein the light is received at the distal end of the optical waveguide and caused by ignition of contamination located at the distal end of the optical waveguide; and
determining that the amount of light satisfies a shutdown threshold.

14. The non-transitory, computer-readable medium of claim 13, wherein the operations further comprise:
determining an intermediary threshold based on laser characteristics or lasing parameters for a surgical procedure, wherein the intermediary threshold is less than the shutdown threshold;
wherein determining the amount of time for stopping the laser beam is based on the intermediary threshold and the amount of light.

15. The non-transitory, computer-readable medium of claim 14, wherein the laser characteristics comprise two or more of (i) an inner diameter of the optical waveguide, (ii) an outer diameter of the optical waveguide, and (iii) an amount of noise expected when detecting the amount of light.

16. The non-transitory, computer-readable medium of claim 14, wherein the lasing parameters comprise two or more of (i) an indication of whether the optical waveguide will be in an open-air environment or a saline environment during the surgical procedure, (ii) an indication of whether the surgical procedure will involve a doping material, and (iii) an indication of a tissue type for lasing during the surgical procedure.

17. The non-transitory, computer-readable medium of claim 14, wherein the operations further comprise:
determining an amount of time between the amount of light satisfying the intermediary threshold and the amount of light satisfying the shutdown threshold;
wherein determining the amount of time for stopping the laser beam is based on the amount of time between the amount of light satisfying the intermediary threshold and the amount of light satisfying the shutdown threshold.

18. The non-transitory, computer-readable medium of claim 14, wherein the operations further comprise:
determining an area between the intermediary threshold and the amount of light as a function of time, bounded by the amount of light satisfying the intermediary threshold and the amount of light satisfying the shutdown threshold;
wherein determining the amount of time for stopping the laser beam is based on the area between the intermediary threshold and the amount of light as a function of time, bounded by the amount of light satisfying the intermediary threshold and the amount of light satisfying the shutdown threshold.

19. The non-transitory, computer-readable medium of claim 11, wherein the laser beam comprises monochromatic light with a power output of approximately 30 watts and a wavelength of approximately 455 nanometers.

20. A computer-implemented method for operating a laser device as an optical plasma drill, the method comprising:
causing a laser source to emit a laser beam;
detecting a plasma ball at a distal end of an optical waveguide, wherein the optical waveguide comprises a proximal end and the distal end, and the optical waveguide is positioned to receive the laser beam from the laser source at the proximal end and emit the laser beam at the distal end;
responsive to detecting the plasma ball, (i) determining an amount of time for stopping the laser beam and (ii) causing the laser source to stop emitting the laser beam for the amount of time; and
causing the laser source to emit the laser beam after the amount of time.
